(19) United States
(12) Patent Application Publication
(10) Pub. No.: US 2024/0243817 A1
(43) Pub. Date: Jul. 18, 2024

(54) RADIATION IMAGING APPARATUS, RADIATION IMAGING SYSTEM, CONTROL METHOD, AND STORAGE MEDIUM

(71) Applicant: CANON KABUSHIKI KAISHA, Tokyo (JP)

(72) Inventors: HIDEAKI MORITA, Tokyo (JP); ASATO KOSUGE, Kanagawa (JP); TOSHITAKA NORO, Saitama (JP)

(21) Appl. No.: 18/619,665

(22) Filed: Mar. 28, 2024

Related U.S. Application Data
(63) Continuation of application No. PCT/JP2022/035343, filed on Sep. 22, 2022.

(30) Foreign Application Priority Data
Sep. 29, 2021 (JP) 2021-159831

(51) Int. Cl. H04B 15/02; H04W 64/00; H04W 88/10

(52) U.S. Cl. CPC H04B 15/02; H04W 64/006; H04W 88/10

(57) ABSTRACT

A radiation imaging apparatus configured to perform radiation imaging includes a first communication unit configured to transmit a radiographic image with an access point included in a radiation imaging system, a second communication unit configured to perform second wireless communication for transmitting and receiving information for establishing wireless communication with a communication device included in the radiation imaging system, and a control unit configured to control wireless communications of the the first and second communication units. The control unit performs control to change a communication form of the wireless communication of at least one of the first and second communication units based on a communication state in transmission of the radiographic image through the first wireless communication.

### CROSS-REFERENCE TO RELATED APPLICATIONS

This application is a Continuation of International Patent Application No. PCT/JP2022/035343, filed Sep. 22, 2022, which claims the benefit of Japanese Patent Application No. 2021-159831, filed Sep. 29, 2021, both of which are hereby incorporated by reference herein in their entirety.

### BACKGROUND OF THE INVENTION

### Field of the Invention

The present invention relates to a radiation imaging apparatus, a radiation imaging system, a control method, and a storage medium.

### Background Art

In recent years, radiation imaging apparatuses that generate digital radiographic images based on irradiated radiation have become widespread, and thus digitalization of radiation imaging systems has progressed. Digitalization of radiation imaging systems makes it possible to check images immediately after radiation imaging and greatly improves workflow compared with conventional imaging methods using films or computed radiography (CR) apparatuses.

In addition, wireless radiation imaging apparatuses have been developed, which facilitates handling of radiation imaging apparatuses. Such a wireless radiation imaging apparatus is used in a plurality of radiation imaging systems, so that a technique for easily linking the radiation imaging apparatus to the radiation imaging system has been discussed.

For example, according to patent literature 1, a technique is discussed in which a link between a radiation imaging apparatus and an access point is established using a short range wireless communication unit different from a wireless communication unit used for transmitting and receiving a radiographic image.

### CITATION LIST

### Patent Literature

PTL 1: Japanese Patent Laid-Open No. 2011-120885

A short range wireless communication unit used for linking to an access point and a wireless communication unit used for transmitting and receiving a radiographic image may use frequency bands (channels) overlapping with each other for wireless communication. A radiation imaging system discussed in patent literature 1 does not consider radio wave interference and the like in the above-described case, so that if radio wave interference occurs, a communication speed and communication quality may decrease.

### SUMMARY OF THE INVENTION

According to an aspect of the present invention, a radiation imaging apparatus configured to perform radiation imaging, includes a first communication unit configured to transmit a radiographic image captured in the radiation imaging, through first wireless communication with an access point included in a radiation imaging system, a second communication unit configured to perform second wireless communication for transmitting and receiving information for establishing the first wireless communication with a communication device included in the radiation imaging system, and a control unit configured to control the first wireless communication and the second wireless communication.

The control unit performs control to change a communication form of at least one of the first wireless communication or the second wireless communication based on a communication state in transmission of the radiographic image through the first wireless communication.

Further features of the present invention will become apparent from the following description of exemplary embodiments with reference to the attached drawings.

### BRIEF DESCRIPTION OF THE DRAWINGS

FIG. 1 illustrates an example of a configuration of a radiation imaging system according to a first exemplary embodiment.

FIG. 2 illustrates an example of a schematic configuration of main components in a radiation imaging apparatus according to the first exemplary embodiment.

FIG. 3 is a flowchart illustrating operations of the radiation imaging apparatus according to the first exemplary embodiment.

FIG. 4 illustrates a period during which a control unit according to the first exemplary embodiment changes a communication form.

FIG. 5 illustrates a period during which a control unit according to the first exemplary embodiment changes a communication form.

FIG. 6 is a flowchart illustrating operations of the radiation imaging apparatus according to the first exemplary embodiment.

FIG. 7 illustrates a period during which a control unit according to the first exemplary embodiment changes a communication form.

FIG. 8 illustrates an example of a schematic configuration of main components in the radiation imaging apparatus according to the first exemplary embodiment.

FIG. 9 illustrates a period during which a control unit according to a second exemplary embodiment changes a communication form.

### DESCRIPTION OF THE EMBODIMENTS

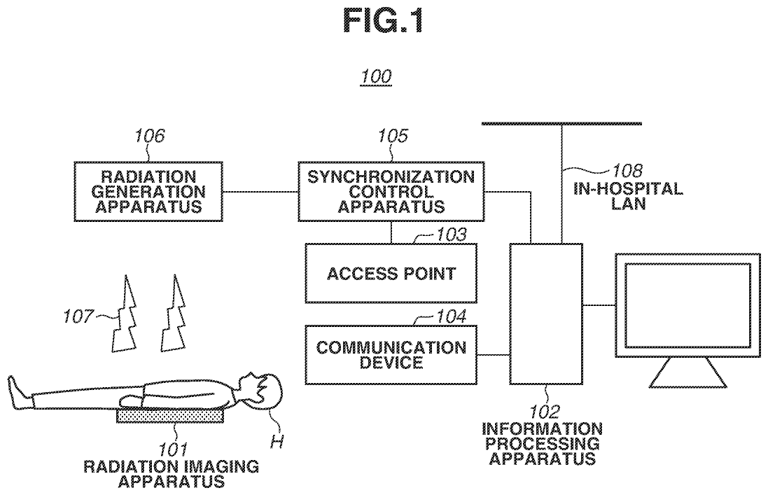

A first exemplary embodiment will be described with reference to FIGS. 1 to 7.

A configuration of a radiation imaging system 100 according to the present exemplary embodiment is initially described with reference to FIG. 1. The radiation imaging system 100 includes a radiation imaging apparatus 101, an information processing apparatus 102, an access point 103, a synchronization control apparatus 105, and a radiation generation apparatus 106.

The radiation imaging apparatus 101 captures a radiographic image based on radiation 107 transmitted through an object H. The radiation imaging apparatus 101 is configured with, for example, a portable radiation imaging apparatus.

The information processing apparatus 102 displays a radiographic image captured by the radiation imaging apparatus 101 on a display unit and instructs an imaging condition input via an operation unit. The information processing apparatus 102 transmits setting information for enabling wireless communication between the radiation imaging apparatus 101 and the information processing apparatus 102.

The access point 103 is a radio wave repeater that wirelessly exchanges information with the radiation imaging apparatus 101.

A communication device 104 is a radio wave transmitter/receiver for performing short range communication between terminals of the radiation imaging apparatus 101 and the information processing apparatus 102. For example, the communication device 104 is a dongle that is connected to the information processing apparatus 102 via a Universal Serial Bus (USB) interface. The communication device 104 may be substituted using a function incorporated in another device, such as the radiation generation apparatus 106.

The communication device 104 complies with at least one of the Bluetooth® Basic Rate/Enhanced Data Rate (BR/EDR) or Bluetooth® Low Energy (LE) standards.

The communication device 104 has a function of a radio frequency identification (RFID) device that exchanges information from a tag embedded with identification (ID) information through short range wireless communication using an electromagnetic field and a radio wave. An RFID communication method may be either an electromagnetic induction method or a radio wave method. The communication device 104 may have a function of an access point.

The synchronization control apparatus 105 includes a circuit that mediates communication and monitors states of the radiation imaging apparatus 101 and the radiation generation apparatus 106. For example, the synchronization control apparatus 105 controls irradiation of the radiation 107 from the radiation generation apparatus 106 and imaging of the object H performed by the radiation imaging apparatus 101. The synchronization control apparatus 105 may include a hub for connecting a plurality of network devices.

The radiation generation apparatus 106 includes a radiation tube in which electrons are accelerated by high voltage to collide with an anode to generate the radiation 107, such as an X-ray. The radiation 107 may be any of a, B, y, X, and neutron rays.

An in-hospital local area network (LAN) 108 is a LAN constructed in a hospital. In the radiation imaging system 100 illustrated in FIG. 1, the radiation generation apparatus 106 irradiates the object H, which is a patient, with the radiation 107. The radiation imaging apparatus 101 detects the radiation 107 transmitted through the object H and generates a radiographic image.

The radiation imaging system 100 can perform synchronous imaging and asynchronous imaging. In the synchronous imaging, timing of radiation irradiation and timing of imaging are synchronized by exchanging an electrical synchronization signal between the radiation imaging apparatus 101 and the radiation generation apparatus 106.

In the asynchronous imaging, the radiation imaging apparatus 101 detects incidence of radiation and starts imaging without exchanging an electrical synchronization signal between the radiation imaging apparatus 101 and the radiation generation apparatus 106. In the asynchronous imaging, the synchronization control apparatus 105 is not provided, and if the radiation generation apparatus 106 emits the radiation 107, the radiation imaging apparatus 101 detects the radiation irradiation, accumulates image signals (charges), and generates a radiographic image. In asynchronous imaging, the radiation imaging apparatus 101 may transfer a radiographic image each time an image is captured or may store the captured image therein without transferring it each time an image is captured.

The radiation imaging system 100 can perform imaging under imaging conditions for general radiation imaging. Examples of such general radiation imaging include fluoroscopic imaging, continuous imaging, still image capturing, digital subtraction angiography (DSA) imaging, roadmap imaging, programmed imaging, tomography, and tomosynthesis imaging.

An imaging frame rate, a tube voltage, a tube current, a sensor readout area, a sensor drive binning setting, a collimator aperture setting, and the like are set to each imaging condition, as information related to imaging. The information related to imaging further includes automatic dose control (ADC), automatic exposure control (AEC), a radiation window width, and whether to store captured images in the radiation imaging apparatus 101.

For example, for the fluoroscopic imaging, the radiation generation apparatus 106 generates pulsed radiation and performs imaging in synchronous imaging. At this time, the radiation imaging system 100 makes settings of the sensor readout area and the sensor drive binning as appropriate and performs imaging.

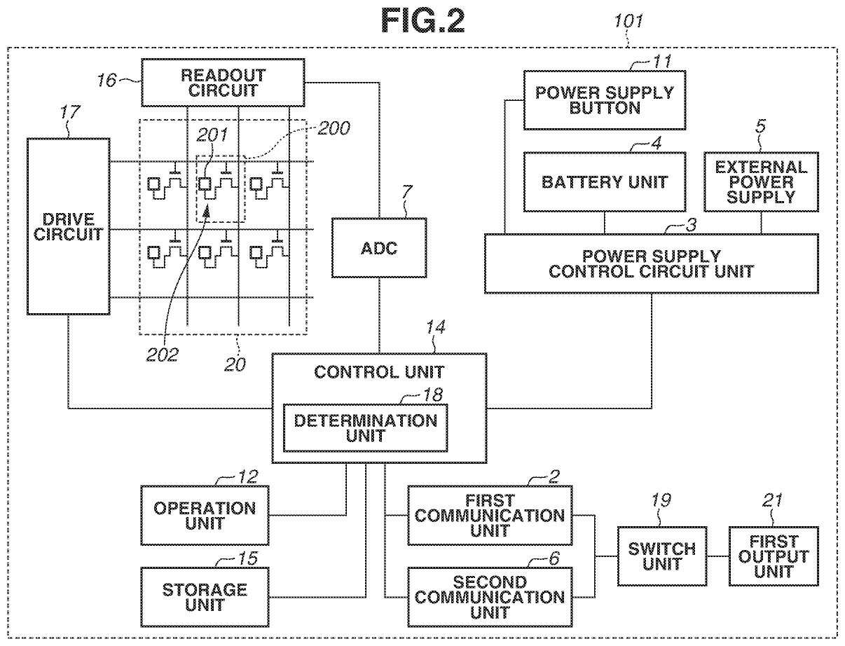

FIG. 2 illustrates an example of a schematic configuration of main components in the radiation imaging apparatus 101 according to the present invention.

A power supply button 11 is operated by the user to start or stop power supply to each component in the radiation imaging apparatus 101. As the power supply button 11, a mechanical switch, a touch panel, and the like may be used. The power supply button 11 is provided, for example, on a side surface of the radiation imaging apparatus 101, but it may be provided at any position on the radiation imaging apparatus 101 as long as it is on a surface other than a radiation incident direction.

A battery unit 4 supplies a predetermined voltage to each unit in the radiation imaging apparatus 101. For example, the battery unit 4 is used to supply power to a control unit 14 and other components, which is described below. A form of the battery unit 4 includes, for example, a lithium ion battery and an electric double layer capacitor, but it may also be implemented using other known techniques. In a case where the radiation imaging apparatus 101 is always supplied with power from an external power supply or the like, the battery unit 4 may be eliminated in the radiation imaging apparatus 101.

An external power supply 5 supplies a predetermined voltage from outside the radiation imaging apparatus 101. A wired power supply method is generally used, but contactless power supply may also be used.

A power supply control circuit unit 3 controls a connection status with the battery unit 4 and the external power supply 5, controls the power supply to each unit in the radiation imaging apparatus 101, and monitors a remaining battery capacity depending on an operation status of the power supply button 11. For example, the power supply control circuit unit 3 transforms a voltage from the battery unit 4 or the like to a predetermined voltage and supplies it to each unit in the radiation imaging apparatus 101. Moreover, in a case where the external power supply 5 is not connected to the radiation imaging apparatus 101, the power supply control circuit unit 3 switches between on and off of the power supply from the battery unit 4 to each unit in the radiation imaging apparatus 101 in response to pressing of the power supply button 11.

A radiation detection unit 20 detects the radiation 107 transmitted through the object H as an image signal (charge). For example, the radiation detection unit 20 includes a photoelectric conversion element and a fluorescent material. The photoelectric conversion element converts light converted by the fluorescent material into an image signal (charge), which is an electrical signal, and stores it.

A drive circuit 17 is an integrated circuit (IC) that provides a drive signal to the radiation detection unit 20 and causes the radiation detection unit 20 to perform operations, such as storing and reading out image signals (charges). More specifically, if the drive circuit 17 selects a pixel 200 in a certain row using the drive signal, a switch element 202 of the pixel 200 in the certain row is sequentially turned ON. The image signals (charges) accumulated in a photoelectric conversion element 201 of the pixel 200 in the certain row is output to a signal line connected to each pixel 200.

A readout circuit 16 has a function of amplifying the image signal (charge) output to the signal line and sequentially reads out the image signal of the radiation detection unit 20. An analog-to-digital converter (ADC) 7 converts an analog image signal read out by the readout circuit 16 into a digital image signal and outputs it to the control unit 14 as a radiographic image. In other words, the ADC 7 is configured as an analog-to-digital (A/D) conversion unit that converts the analog image signal read out by the readout circuit 16 into digital data.

A storage unit 15 stores radiographic image data output from the ADC 7, a system identifier of the radiation imaging system 100 to be linked, a calculated distance threshold value calculated from radio wave intensity between the radiation imaging apparatus 101 and the communication device 104, an offset image, and the like. The storage unit 15 may store the generated image data in association with an engineer ID, which is identification information regarding a corresponding engineer, a patient ID, which is patient identification information, imaging time, an imaging dose, an imaging region, imaging conditions including the number of captured images, a transfer history for radiographic image data, and the like.

A nonvolatile memory, such as a flash memory, is desirably used as the storage unit 15, but the storage unit 15 is not limited to this, and may be a volatile memory, such as a synchronous dynamic random access memory (SDRAM). The storage unit 15 may be configured to be detachable so that it can be attached to and detached from the information processing apparatus 102 or the like.

A wireless communication module is set to a first communication unit 2 in accordance with a medium used for communication in the information processing apparatus 102 and the synchronization control apparatus 105. For example, the first communication unit 2 can communicate with the access point 103 via a wireless LAN (WLAN) and transmit and receive the radiographic image and the like to and from the information processing apparatus 102.

A wireless communication module is set to a second communication unit 6 in accordance with a medium used for communication in the information processing apparatus 102 and the synchronization control apparatus 105. For example, the second communication unit 6 communicates with the communication device 104 via a wireless personal area network (PAN). The second communication unit 6 is able to transmit and receive an identifier of the radiation imaging system 100 and settings, such as a service set identifier (SSID), an encryption key, and an Internet Protocol (IP) address, to be used for establishing communication with the first communication unit 2.

The second communication unit 6 can also transmit information about a position and an orientation of the radiation imaging apparatus 101 to the information processing apparatus 102 and the radiation generation apparatus 106. The information about the position and the orientation are obtainable using an acceleration sensor, a gyro sensor, a geomagnetic sensor, a Global Positioning System (GPS) sensor, and other known techniques. The second communication unit 6 is also able to set an ID to the radiation imaging apparatus 101 for linking the radiographic image to information to be applied to a patient to be imaged.

For example, the communication device 104 is able to transmit to the second communication unit 6 patient information input to the information processing apparatus 102 and patient information acquired with a bar code reader equipped with a communication device that is capable of communicating with the second communication unit 6.

The second communication unit 6 is able to transmit an imaging availability state of the radiation imaging apparatus 101 and a state of the battery unit 4 to an external device such as the information processing apparatus 102 and the radiation generation apparatus 106.

For example, the imaging availability state indicates whether the radiation imaging apparatus 101 is in a state in which the radiation detection unit 20, the readout circuit 16, and the drive circuit 17 are powered on, readout and other preparation operations are completed, and an analog image signal read out by the readout circuit 16 is convertible into a digital image signal. Further, the state of the battery unit 4 indicates a state related to a charging status of the battery unit 4. For example, whether power is supplied to the radiation imaging apparatus 101, a remaining charge value of the battery unit 4, and whether the remaining charge value of the battery unit 4 is a certain value or less are indicated.

A switch unit 19 switches transmission and reception data of the first communication unit 2 or the second communication unit 6. For example, the switch unit 19 is configured with a switching IC, such as an analog switch IC, and enables communication of transmission and reception data of the first communication unit 2 and the second communication unit 6 in a time-division manner.

A first output unit 21 converts communication data transmitted by the switch unit 19 into a radio wave and transmits it. The first output unit 21 converts radio waves transmitted by the access point 103 and/or the communication device 104 into communication data. For example, the first output unit 21 includes a wireless communication antenna such as a plane antenna or a dipole antenna.

An operation unit 12 is a button used as a manual trigger for transferring the setting information between the radiation imaging apparatus 101 and the communication device 104. The operation unit 12 may be a mechanical switch, a touch panel, or the like may be used. When operated, the operation unit 12 may be able to transmit and receive the identifier of the radiation imaging system 100, the SSID and the encryption key set to the first communication unit 2, and the like. The operation unit 12 is provided on the side surface of the radiation imaging apparatus 101, but may be provided at any position as long as it is on the surface other than the radiation incident direction.

The control unit 14 controls the units of the radiation imaging apparatus 101. Due to the nature of the radiation imaging apparatus 101, the control unit 14 is demanded to be small in size, lightweight, and to have a power-saving feature. In order to achieve these requirements, a field programmable gate array (FPGA) or a dedicated IC circuit may be used for the control unit 14.

The control unit 14 includes a determination unit 18. The determination unit 18 determines a state of communication of the radiographic image which is performed by the first communication unit 2 when the second communication unit 6 performs wireless communication. For example, in a case where the first communication unit 2 performs wireless communication at the same time as the second communication unit 6 performs wireless communication, radio wave interference may occur. Thus, according to the present exemplary embodiment, a communication form of at least one of the wireless communication performed by the first communication unit 2 and the wireless communication performed by the second communication unit 6 is changed depending on a state of the wireless communication performed by the first communication unit 2.

The communication state of a radiographic image refers to a state in which the radiographic image is being communicated or a state in which the communication of the radiographic image is performable. For example, the determination unit 18 determines whether the radiographic image is being communicated from information indicating whether there is a radiographic image transmission buffer, whether a radiographic image communication protocol (e.g., Transmission Control Protocol/Internet Protocol (TCP/IP)) is connected, and the like. The determination unit 18 determines whether the transmission of a radiographic image is performable based on whether the imaging preparation of the radiation detection unit 20 is completed or whether the radiation imaging apparatus 101 receives an instruction to shift to imaging executable state, from the information processing apparatus 102 and becomes ready for imaging in synchronization with the radiation generation apparatus 106.

The control unit 14 changes the communication form of the first communication unit 2 or the second communication unit 6 based on a determination result of the determination unit 18. For example, in a case where the determination unit 18 determines that the radiographic image is being communicated using the first communication unit 2 when the second communication unit 6 communicates the identifier of the radiation imaging system 100, the control unit 14 stops communication of the second communication unit 6. The control unit 14 may stop communication by stopping the power supply to the second communication unit 6, discarding a communication packet and/or connection, and the like.

As an example of a change in the communication form of the control unit 14, the control unit 14 may change a radio frequency used by the first communication unit 2 to a frequency that is not used by the second communication unit 6. For example, in a case where the first communication unit 2 transmits a radiographic image using WLAN and radio waves in a 2.4 GHz band, and the second communication unit 6 performs communication using Bluetooth® and radio waves in the 2.4 GHz band, the control unit 14 changes the radio frequency of WLAN to a 5 GHz band.

Further, as another example of the change in the communication form of the control unit 14, the control unit 14 may prioritize communication of the first communication unit 2 over that of the second communication unit 6. For example, in a case where a communication packet of the second communication unit 6 and a communication packet of a radiographic image transmitted by the first communication unit 2 are input to a transmission buffer, the control unit 14 may transmit the communication packet to be transmitted by the first communication unit 2 before transmission of the communication packet which is to be performed by the second communication unit 6.

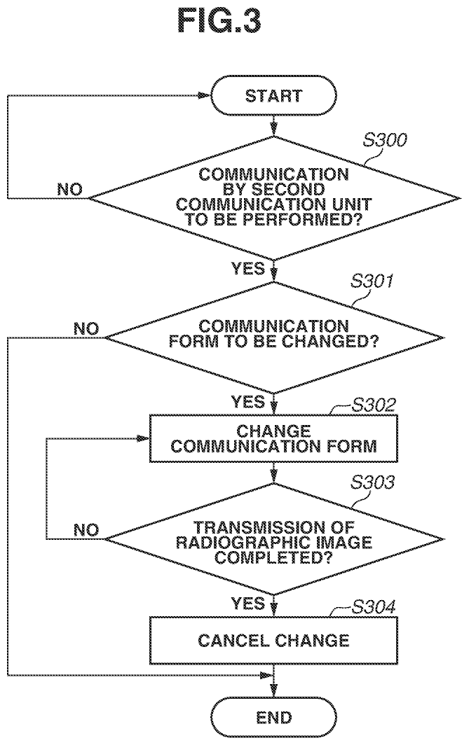

FIG. 3 is a flowchart illustrating a method for controlling the radiation imaging apparatus 101 according to the present invention.

In step S300, the determination unit 18 determines whether to perform wireless communication with the second communication unit 6, based on a status of a transmission/reception communication buffer and a state of a communication controller. In a case where the control unit 14 is set in advance to perform communication with the second communication unit 6 through an operation of the operation unit 12, it is also possible to determine whether the operation unit 12 has been operated. If it is determined that the second communication unit 6 performs the wireless communication (YES in step S300), the processing proceeds to step S301. If it is determined that the second communication unit 6 does not perform communication (NO in step S300), the determination unit 18 performs the operation in step S300 again.

In step S301, the determination unit 18 determines whether the state of communication of the radiographic image performed by the first communication unit 2 is a state in which the communication form of the first communication unit 2 or the second communication unit 6 is to be changed when the second communication unit 6 performs communication. If the determination unit 18 determines that the communication form is to be changed (YES in step S301), the processing proceeds to step S302. If not (NO in step S301), the processing of the flowchart is terminated, and the second communication unit 6 executes wireless communication.

In step S302, the control unit 14 changes the communication form of the wireless communication using the first communication unit 2 or the second communication unit 6 based on a determination result of the determination unit 18. The processing then proceeds to step S303.

In step S303, the determination unit 18 determines whether the first communication unit 2 completes the transmission of the radiographic image or whether it is no longer in a transmission-performable state. If the determination unit 18 determines that the first communication unit 2 completes the transmission or that it is no longer in a transmission-performable state (YES in step S303), the processing proceeds to step S304. Otherwise (NO in step S303), the processing returns to step S302. In other words, the control unit 14 repeats the operations in steps S302 and S303 until the determination unit 18 determines that the state in which the first communication unit 2 is performing wireless communication or the state in which the wireless communication is possible is terminated.

In step S304, the control unit 14 cancels the change in the communication form of the wireless communication using the first communication unit 2 or the second communication unit 6, which has been changed in step S302, and changes the communication form again to the communication form of the wireless communication before the change in step S302.

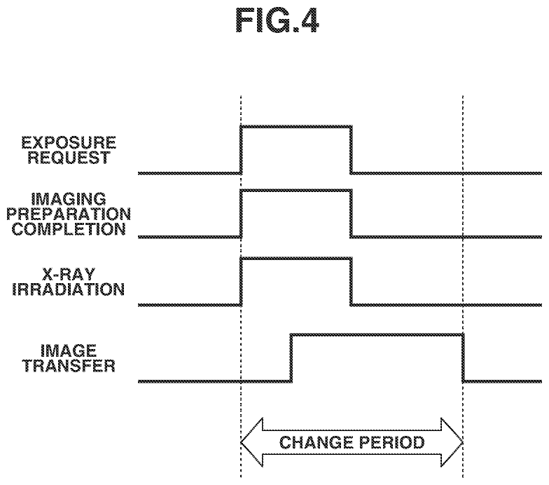

FIG. 4 illustrates an example of a period during which the control unit 14 changes the communication form in a case where the determination unit 18 of the radiation imaging apparatus 101 according to the present invention determines that the radiation imaging apparatus 101 is in a state in which a radiographic image is transmittable.

The radiation generation apparatus 106 issues an exposure request to the radiation imaging apparatus 101 via the synchronization control apparatus 105. The radiation imaging apparatus 101 receives an imaging preparation instruction from the information processing apparatus 102, and if the imaging preparation is completed, notifies the radiation generation apparatus 106 via the synchronization control apparatus 105 that exposure is possible.

The radiation generation apparatus 106 emits radiation in response to receiving the notification that exposure is possible, from the radiation imaging apparatus 101. In a case where the notification that exposure is possible is issued via the synchronization control apparatus 105, the determination unit 18 determines that the radiation imaging apparatus 101 is in a state in which the radiation image is transmittable. Further, in a case where the determination unit 18 determines that communication is performed by the second communication unit 6, the control unit 14 restricts the communication of the first communication unit 2 or the second communication unit 6. If the determination unit 18 determines that the transmission of the radiographic image is completed, the control unit 14 performs control to cancel communication restriction.

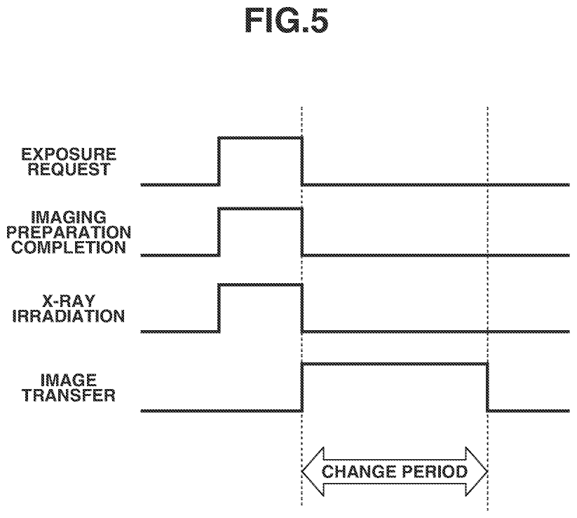

FIG. 5 illustrates an example of a period during which the control unit 14 changes the communication form in a case where the determination unit 18 of the radiation imaging apparatus 101 according to the present invention determines that a radiographic image is being transmitted. Operations from when the radiation generation apparatus 106 issues an exposure request to when radiation is emitted are similar to those in FIG. 4.

The determination unit 18 determines whether the radiographic image is being transmitted using the first communication unit 2. Further, in a case where the determination unit 18 determines that communication is to be performed by the second communication unit 6, the control unit 14 restricts the communication of the first communication unit 2 or the second communication unit 6. If the determination unit 18 determines that the transmission of the radiographic image is completed, the control unit 14 performs control to cancel the communication restriction.

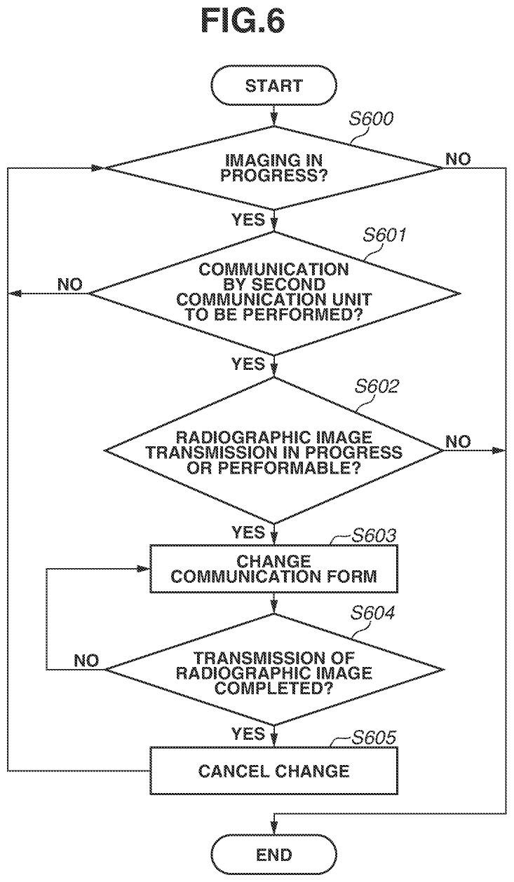

FIG. 6 is a flowchart illustrating a method for controlling the radiation imaging apparatus 101 in a case where a radiographic image is transmitted a plurality of times in imaging, such as fluoroscopy or image division transmission.

In step S600, the determination unit 18 determines whether imaging instructed by the information processing apparatus 102 is being performed to the radiation imaging apparatus 101, and in a case where imaging is in progress (YES in step S600), the processing proceeds to step S601. In a case where imaging is not in progress (NO in step S600), the processing in the flowchart is terminated. For example, in a case where a desired number of radiographic images set from the information processing apparatus 102 to the radiation imaging apparatus 101 is transmitted from the first communication unit 2, the determination unit 18 determines that imaging is completed and is not in progress.

A transmission method of a radiographic image is to divide one frame of a radiographic image generated by the radiation detection unit 20 and transmit it from the first communication unit 2. One frame is divided in such a manner that the drive circuit 17 and the readout circuit 16 thin out the pixels 200 arranged in the radiation detection unit 20 and generate digital signals from the ADC 7 to the control unit 14. The first communication unit 2 may perform communication after the control unit 14 thins out one frame of the radiographic image.

As another example of a method for determining whether imaging is in progress, the determination unit 18 may determine whether there is an exposure request from the radiation generation apparatus 106 or whether a display or a state of the information processing apparatus 102 indicating that imaging is in progress.

In step S601, the determination unit 18 determines whether to perform wireless communication with the second communication unit 6 based on the status of the transmission/reception communication buffer and the state of the communication controller. In a case where the control unit 14 is set in advance to perform communication by the second communication unit 6 with an operation of the operation unit 12, it is also possible to determine whether the operation unit 12 is operated. If the determination unit 18 determines to perform the communication with the second communication unit 6 (YES in step S601), the processing proceeds to step S602. If the determination unit 18 determines not to perform the communication with the second communication unit 6 (NO in step S601), the determination unit 18 performs the operation in step S600 again.

In step S602, the determination unit 18 determines the communication state of the radiographic image by the first communication unit 2. In a case where the first communication unit 2 is in a state in which the radiographic image is being transmitted or in a radiographic-image-transmission performable state (YES in step S602), the processing proceeds to step S603. In a case where the first communication unit 2 is not in the state in which the radiographic image is being transmitted or in the radiographic-image-transmission performable state (NO in step S602), the processing in the present flowchart is terminated, and the second communication unit 6 executes wireless communication.

In step S603, the control unit 14 changes the communication form of the first communication unit 2 or the second communication unit 6 based on a result of the determination unit 18, and the processing proceeds to step S604.

In step S604, the determination unit 18 determines whether the first communication unit 2 completes the transmission of the radiographic image or whether it is no longer in the radiographic-image-transmission performable state. In a case where the determination unit 18 determines that the first communication unit 2 completes the transmission or the first communication unit 2 is no longer in the radiographic-image-transmission performable state (YES in step S604), the processing proceeds to step S605. Otherwise (NO in step S604), the processing returns to step S603. In other words, the control unit 14 repeats the operations in steps S603 and S604 until the determination unit 18 determines that the state in which the first communication unit 2 is performing the wireless communication or the state in which the wireless communication is performable is terminated.

In step S605, the control unit 14 cancels the change in the communication form of the wireless communication of the first communication unit 2 or the second communication unit 6 changed in step S603 and changes it again to the communication form of the wireless communication before the change in step S603. The processing returns to step S600, the operations in steps S600 to S605 are repeated until the imaging is completed, and if the imaging is completed, the processing in the flowchart is terminated based on a determination made in step S600.

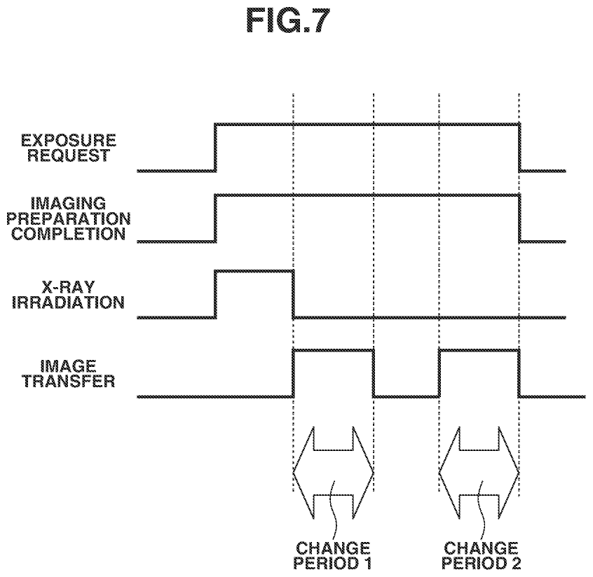

FIG. 7 illustrates an example of a period during which the control unit 14 changes the communication form in a case where a radiographic image is transmitted a plurality of times in imaging, such as fluoroscopy or image division transmission. Operations from when the radiation generation apparatus 106 issues an exposure request to when radiation is emitted are similar to those in FIG. 4.

In a case where the radiation generation apparatus 106 issues an exposure request to the radiation imaging apparatus 101, the determination unit 18 determines that imaging is in progress. The determination unit 18 further determines whether the radiographic image is being transmitted using the first communication unit 2. Further, in a case where the determination unit 18 determines that communication is to be performed by the second communication unit 6, the control unit 14 changes the communication form of the first communication unit 2 or the second communication unit 6 (a change period 1).

If the determination unit 18 determines that transmission of the radiographic image is completed, the control unit 14 performs control to cancel the change in the communication form. In a case where the radiation generation apparatus 106 issues the exposure request to the radiation imaging apparatus 101, the determination unit 18 determines that imaging is in progress and determines whether the radiographic image is being transmitted using the first communication unit 2. Then, processing similar to that in the change period 1 is performed (a change period 2).

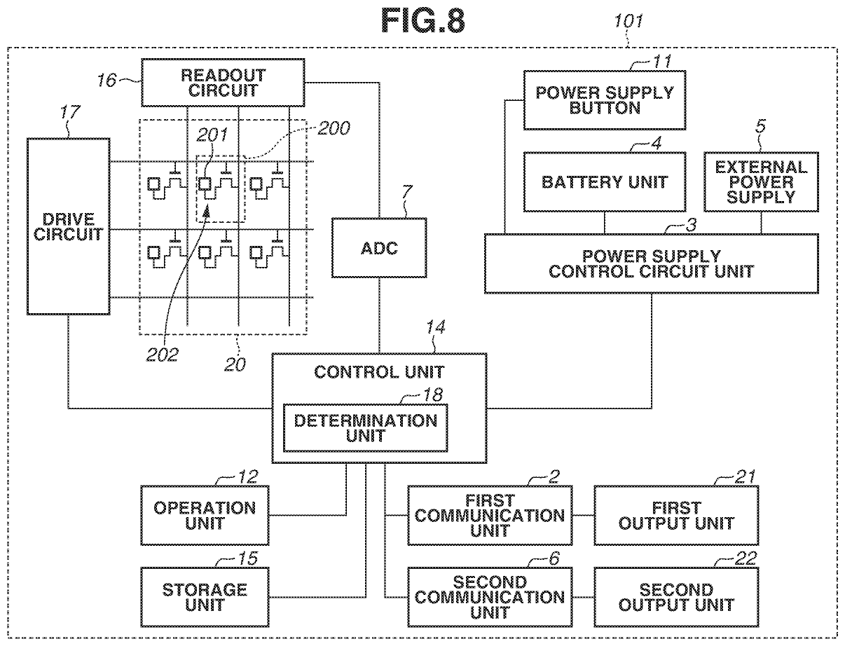

In the above description, the first communication unit 2 and the second communication unit 6 are connected to the same output unit, namely, the first output unit 21, via the switch unit 19, but the present invention is not limited to this configuration. As illustrated in FIG. 8, the first communication unit 2 and the second communication unit 6 may be provided with the first output unit 21 and a second output unit 22, which are respectively corresponding output units.

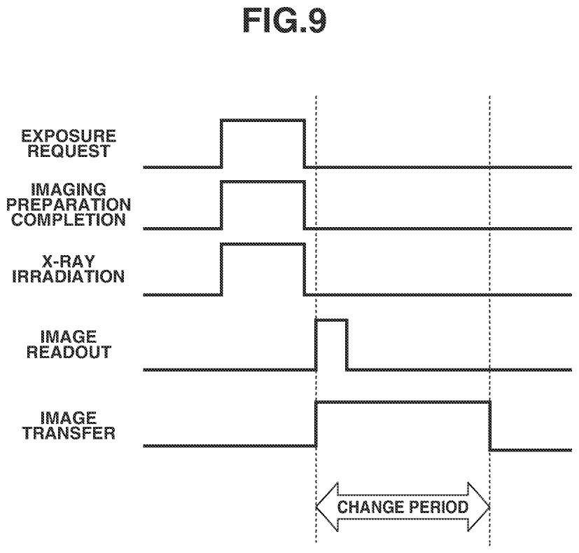

A second exemplary embodiment of the present invention will be described. FIG. 9 illustrates an example of a period during which the control unit 14 changes the communication form in a state in which a radiographic image is going to be transmitted. Here, a description will be provided of an operation example in a case where an operation of reading out an image signal (charge) is determined to be a state in which a radiation image is going to be transmitted. An example of operations from when the radiation generation apparatus 106 issues an exposure request to when the X-ray is emitted is similar to that illustrated in FIG. 4.

If communication is performed around the radiation imaging apparatus 101 at a time of reading out image signals, generation of the image signals (charges) may be affected, and noise may occur in an image. Thus, according to the present exemplary embodiment, wireless communication of the second communication unit 6 is stopped in this period.

The determination unit 18 provides a drive signal to the radiation detection unit 20 using the drive circuit 17 and determines whether an operation of reading out the image signal (charge) is started. In a case where the determination unit 18 determines whether the radiographic image is being transmitted using the first communication unit 2, the control unit 14 stops wireless communication performed by the second communication unit 6.

If the determination unit 18 determines that transmission of the radiographic image is completed, the control unit 14 performs control to restore the changed communication form to its original state. Communication during readout is stopped using the above-described unit, so that it is possible to prevent or reduce an influence on image noise during reading out the image signal. A state in which the radiographic image is going to be transmitted may include a state in which the drive circuit 17 provides a drive signal to the radiation detection unit 20 and accumulates image signals and/or an idle reading operation, which is a readout operation in a state in which no radiation irradiation occurs.

The present invention is not limited to the above-described exemplary embodiments, and various modifications and changes can be made without departing from the spirit and the scope of the present invention. Therefore, the following claims are attached in order to publicize the scope of the present invention.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

A communication speed and communication quality in transmitting a radiographic image are improved in a radiation imaging apparatus in which a plurality of communication units each performs a link operation and communication of a radiographic image.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging apparatus configured to perform radiation imaging, the radiation imaging apparatus comprising:
a first communication unit configured to transmit a radiographic image captured in the radiation imaging, through first wireless communication with an access point included in a radiation imaging system;
a second communication unit configured to perform second wireless communication for transmitting and receiving information for establishing the first wireless communication with a communication device included in the radiation imaging system; and
a control unit configured to control the first wireless communication and the second wireless communication,
wherein the control unit performs control to change a communication form of at least one of the first wireless communication or the second wireless communication based on a communication state in transmission of the radiographic image through the first wireless communication.

2. The radiation imaging apparatus according to claim 1, wherein, in a case where the transmission of the radiographic image is being performed through the first wireless communication or in a case where the transmission of the radiographic image is performable thorough the first wireless communication, the control unit performs control to change the communication form of at least one of the first wireless communication or the second wireless communication.

3. The radiation imaging apparatus according to claim 2, wherein, in a case where the transmission of the radiographic image is being performed through the first wireless communication or in a case where the transmission of the radiographic image is performable through the first wireless communication, the control unit performs control to change the communication form of at least one of the first wireless communication or the second wireless communication in transmitting, with the second communication unit, information about at least one of a position and an orientation of the radiation imaging apparatus with the communication device.

4. The radiation imaging apparatus according to claim 2, wherein, in a case where the transmission of the radiographic image is being performed through the first wireless communication or in a case where the transmission of the radiographic image is performable through the first wireless communication, the control unit performs control to change the communication form of at least one of the first wireless communication or the second wireless communication in transmitting, with the second communication unit, identification information about an object for linking the radiographic image to information about the object captured in the radiographic image.

5. The radiation imaging apparatus according to claim 2, wherein, in a case where the transmission of the radiographic image is being performed through the first wireless communication or in a case where the transmission of the radiographic image is performable through the first wireless communication, the control unit performs control to change the communication form of at least one of the first wireless communication or the second wireless communication in transmitting, through the second communication unit, information indicating a imaging availability state of the radiation imaging apparatus.

6. The radiation imaging apparatus according to claim 2, further comprising a battery unit configured to supply power to each unit in the radiation imaging apparatus,
wherein, in a case where the transmission of the radiographic image is being performed through the first wireless communication or in a case where the transmission of the radiographic image is performable through the first wireless communication, the control unit performs control to change the communication form of at least one of the first wireless communication or the second wireless communication in transmitting, with the second communication unit, information indicating a state of the battery unit.

7. The radiation imaging apparatus according to claim 1, wherein the control unit performs control to stop the second wireless communication.

8. The radiation imaging apparatus according to claim 1, wherein the control unit performs control to change a radio frequency to be used in the first wireless communication to a radio frequency that is not used in the second wireless communication.

9. The radiation imaging apparatus according to claim 1, wherein the control unit performs control to prioritize the first wireless communication over the second wireless communication.

10. The radiation imaging apparatus according to claim 1, wherein the control unit performs control to further change the changed communication form of the first wireless communication or the second wireless communication to the communication form before the change in response to detection of completion of the transmission of the radiographic image through the first wireless communication.

11. The radiation imaging apparatus according to claim 1, wherein the first communication unit is a local area network (LAN).

12. The radiation imaging apparatus according to claim 1, wherein the second communication unit is a personal area network (PAN).

13. A radiation imaging system comprising:
a radiation imaging apparatus configured to perform radiation imaging, the radiation imaging system comprising:
a first communication unit configured to transmit a radiographic image captured in the radiation imaging, through first wireless communication with an access point included in a radiation imaging system;
a second communication unit configured to perform second wireless communication for transmitting and receiving information for establishing the first wireless communication with a communication device included in the radiation imaging system; and
a control unit configured to control the first wireless communication and the second wireless communication,
wherein the control unit performs control to change a communication form of at least one of the first wireless communication or the second wireless communication based on a communication state in transmission of the radiographic image through the first wireless communication;
the access point; and
the communication device.

14. A method for controlling a radiation imaging apparatus configured to perform radiation imaging and including a first communication unit configured to transmit a radiographic image through first wireless communication with an access point included in a radiation imaging system, a second communication unit configured to perform second wireless communication for transmitting and receiving information for establishing the first wireless communication with a communication device included in the radiation imaging system, and a control unit configured to control the first wireless communication and the second wireless communication, the method comprising,
performing control to change a communication form of at least one of the first wireless communication or the second wireless communication based on a communication state in transmission of the radiographic image through the first wireless communication.

15. A non-transitory computer-readable storage medium storing a program for causing a computer to perform the control method according to claim 14.
